(19) United States
(12) Patent Application Publication
(10) Pub. No.: US 2016/0281001 A1
(43) Pub. Date: Sep. 29, 2016

(54) FLAME RETARDANT COMPOSITION

(71) Applicant: THOR GMBH, Speyer (DE)

(72) Inventor: Volker BUTZ, Neustadt/Weinstrasse (DE)

(21) Appl. No.: 15/022,715

(22) PCT Filed: Sep. 10, 2014
(86) PCT No.: PCT/EP2014/002445

§ 371 Date: Mar. 17, 2016

(30) Foreign Application Priority Data
Sep. 17, 2013 (EP) 13004537.0

(51) Int. Cl. C09K 21/12; C07D 251/54; C08J 9/40; C08K 5/3492; C08J 5/18; C07F 9/40; C08K 5/5317

(52) U.S. Cl. CPC C09K 21/12; C07F 9/4068; C07D 251/54; C08K 5/5317; C08K 5/34922; C08K 5/34928; C08J 5/18; C08J 9/40; C08J 2323/06

(57) ABSTRACT

The invention relates to a flame retardant composition comprising a salt of components (A) and (B), wherein (A) is at least one pyrophosphonic acid derivative of the general formula (I):

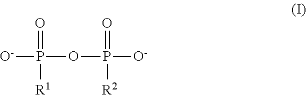

where R¹ and R² are each independently a substituted or unsubstituted, straight-chain or branched alkyl group having 1 to 4 carbon atoms, substituted or unsubstituted benzyl or substituted or unsubstituted phenyl, and (B) is selected from the group consisting of melamine, melam, melem, melon, ammeline, ammelide, 2-ureidomelamine, substituted or unsubstituted heptazine, guanylurea, acetoguanamine, benzoguanamine and diaminophenyltriazine or two or more of these compounds.

The invention relates to a flame retardant composition comprising a salt of components (A) and (B), wherein (A) is at least one pyrophosphonic acid derivative of the general formula I:

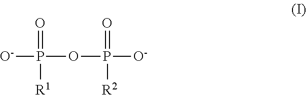

where R¹ and R² are each independently a substituted or unsubstituted, straight-chain or branched alkyl group having 1 to 4 carbon atoms, substituted or unsubstituted benzyl or substituted or unsubstituted phenyl, and (B) is selected from the group consisting of melamine, melam, melem, melon, ammeline, ammelide, 2-ureidomelamine, substituted or unsubstituted heptazine, guanylurea, acetoguanamine, benzoguanamine and diaminophenyltriazine or two or more of these compounds.

The use of the combination of melamine condensation products and phosphorus compounds in flame retardant compositions is known, for example, from DE 601 11 720 T2. This reference describes, for example, salts of melamine condensation products with a phosphorus-containing acid, the phosphorus-containing acid being an acid having just one acid equivalent, and the melamine condensation product being melam, for example. However, a disadvantage with regard to these combinations is that they have a tendency to side reactions, for example at elevated temperatures. In particular, the phosphorus esters can lead to alkylation of the nitrogen component.

WO 00/02869 A1 discloses polyphosphate salts of 1,3,5-triazine compounds having a mean degree of condensation (number-average) greater than 20 and a molar ratio of triazine compound, such as of melamine, to phosphorus (M/P) >1.1. This document additionally also describes a two-stage process for preparing these salts by conversion of a 1,3,5-triazine compound with orthophosphoric acid to the corresponding orthophosphate salt, and thermal treatment for conversion of the orthophosphate salt to a polyphosphate of the 1,3,5-triazine compound. In addition to the orthophosphates, it is also possible to use pyrophosphates. The polyphosphate salts described in this publication are appropriately to be used as flame retardants. However, phosphates or polyphosphate are comparatively weak flame retardants and are still unable to develop their flame retardancy particularly at relatively low temperatures.

WO 96/09344 describes the use of melamine- or melem-phosphoric acid reaction products as flame retardants in glass fiber-reinforced polyamide molding compounds. Co-flame retardants used are zinc borate, zinc phosphate, etc. These flame retardants may still have almost satisfactory flame retardancy in polyamides, but still do not exhibit satisfactory efficacy in polyolefins in particular.

EP 1 544 206 A1 describes dialkylphosphinic acids and salts thereof, which are used together with further selected components as flame retardants in thermoplastic polymers. According to this prior art, a particularly suitable combination is one of aluminum trisdiethylphosphinate, melamine polyphosphate, zinc oxide and glass fibers in nylon-6,6. These flame retardants too may still have almost satisfactory flame retardancy in polyamides, but still do not exhibit satisfactory efficacy in polyolefins in particular.

Document EP 0 363 321 A1 describes melamine salts of methylphosphonic acid as flame retardants. The melamine salts can be prepared by reaction of methylphosphonic acid or the monomethyl ester thereof with melamine. The disadvantage of these salts is that they are comparatively hygroscopic, and still do not have satisfactory thermal stability.

Proceeding from the prior art, it is an object of the invention to provide a flame retardant based on melamine derivatives and a phosphorus-containing compound, which can be produced in an economically viable manner. It is a particular object of the invention to provide a flame retardant which has high thermal stability and is hygroscopic only to a small degree. It is a further object of the invention to provide a flame retardant which has high efficacy in the polymer, especially in polyolefins, and can therefore be incorporated in smaller amounts, as a result of which the properties of the polymer are affected to a smaller degree.

This object is achieved by a flame retardant composition comprising a salt of components (A) and (B), wherein:

(A) is at least one alkylpyrophosphonic acid derivative of the general formula (I):

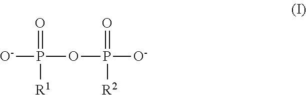

where R¹ and R² are each independently a substituted or unsubstituted, straight-chain or branched alkyl group having 1 to 4 carbon atoms, substituted or unsubstituted benzyl or substituted or unsubstituted phenyl, and

(B) is selected from the group consisting of melamine, melam, melem, melon, ammeline, ammelide, 2-ureidomelamine, substituted or unsubstituted heptazine, guanylurea, acetoguanamine, benzoguanamine and diaminophenyltriazine or two or more of these compounds.

It is a feature of the above-defined flame retardant composition that the salt of components (A) and (B) present therein has a high thermal stability up to 290° C., as a result of which the flame retardant composition of the present invention can be incorporated at high temperatures into polymers such as polyamide, polybutyl terephthalate (PBT), polystyrene (PS), high-impact polystyrene (HIPS) and acrylonitrile-butadiene-styrene (ABS) without breaking down. Further advantages are that the salt of components (A) and (B) is hygroscopic only to a small degree, if at all. A further advantage lies in the processibility of the flame retardant composition of the invention, for example with respect to the compositions described in EP 0 363 321 A1, since compositions of this kind can release water and/or alcohols, for example, on incorporation into the polymer and, in the case of use of an extruder in the incorporation into a polymer or the processing of a polymer modified with the flame retardant composition of the invention, leads to a relatively low level of deposits on the extruder screw. The monoesters described in EP 0 363 321 A1 can lead, for example, to alkylation reactions, which gives rise to odorous and in some cases toxic compounds. The components of the flame retardant composition of the invention additionally have low water solubility, which means that they are leached out of the polymer to a smaller degree on contact with water or moisture. The polymers modified with the flame retardant composition of the invention are therefore particularly suitable for agricultural films and moldings, housings or cables for moisture-prone rooms, washing machines and motor vehicles.

In a further embodiment of the invention, the above-defined flame retardant composition comprises, as a further component, component (C), one or more component(s) that act(s) synergistically with components (A) and (B). In this embodiment of the invention, this component (C) is a hindered amine compound sold under the FLAMESTAB® NOR 116 brand name by BASF SE. This flame retardant is disclosed in EP 0 889 085, the disclosure of which is included in the present application by reference. Alternatively or additionally, component (C) is a poly[2,4-(piperazin-1,4-yl)-6-(morpholin-4-yl)-1,3,5-triazine], which is also commercially available under the ppmTriazin® brand name.

The proportion of this component (C), based on the sum total of all components of the flame retardant composition, is generally in the range from 1% to 30% by weight, preferably in the range from 5% to 25% by weight, more preferably in the range from 10% to 20% by weight.

Component (A) of the general formula (I) is a pyrophosphonic acid derivative in which R¹ and R² are each independently a substituted or unsubstituted, straight-chain or branched alkyl group having 1 to 4 carbon atoms, substituted or unsubstituted benzyl or substituted or unsubstituted phenyl. In a preferred embodiment of the invention, R¹ and R² are the same and are selected from the group of methyl, ethyl, propyl and phenyl.

“Alkyl” means a saturated aliphatic hydrocarbon group which may be straight-chain or branched and may have from 1 to 4 carbon atoms in the chain. Alkyl is, for example, methyl, ethyl, 1-propyl, 2-propyl, 1-butyl, 2-butyl, 2-methyl-1-propyl (isobutyl) or 2-methyl-2-propyl (tert-butyl), preferably methyl.

“Substituted alkyl”, or “substituted benzyl” or “substituted phenyl”, means that the alkyl group or benzyl or phenyl group is substituted by one or more substituents selected from alkyl, optionally substituted aryl, optionally substituted aralkyl, alkoxy, amino, nitro, carboxyl, carboalkoxy, cyano, alkylamino, halo, hydroxyl, hydroxyalkyl, mercaptyl, alkylmercaptyl, trihaloalkyl, carboxyalkyl and carbamoyl.

In a preferred embodiment of the invention, component (A) is pyromethanephosphonic acid, an alkylpyrophosphonic acid derivative of the formula (II):

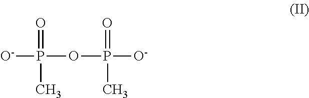

Component (B) of the present invention is at least one compound selected from the group consisting of melamine, melam, melem, melon, ammeline, ammelide, 2-ureidomelamine, substituted or unsubstituted heptazine, guanylurea, acetoguanamine, benzoguanamine and diaminophenyltriazine or two or more thereof.

In one embodiment of the invention, component (B) is a substituted or unsubstituted heptazine derivative of the general formula (III):

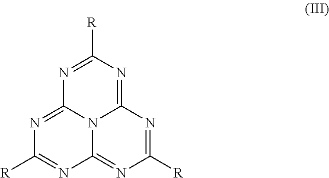

In this formula, R are, for example, independently H, or NH₂. Compounds of this kind are known to those skilled in the art, for example from WO 2006/034784 A1 or EP 2 256 122 A1, the disclosure of which is incorporated here in full by reference.

Component (B), in a preferred embodiment of the invention, is melamine.

Salts preferred in accordance with the invention are the melamine salt of pyromethylphosphonic acid or the melamine salt of pyroethylphosphonic acid or the melamine salt of pyropropylphosphonic acid or the melamine salt of pyrophenylphosphonic acid. Particular preference is given to the melamine salt of pyromethylphosphonic acid.

The molar ratio of components (A) to (B) relative to one another varies depending on the component(s) (B). If component (B) is a compound selected from the group of melam, melem, melon, ammeline, ammelide, 2-ureidomelamine, substituted or unsubstituted heptazine, guanylurea, acetoguanamine, benzoguanamine and diaminophenyltriazine or two or more thereof, the molar ratio of (A) to (B) is 0.5:2 to 2:0.5, preferably 1:1. If component (B) is melamine, the molar ratio of (A) to (B) is 1:2. The exact molar ratio of (A) to (B) depends particularly on whether component (B) is mono- or polybasic. The molar ratio of components (A) and (B) relative to one another should generally be chosen such that all the free acid groups of component (A) are satisfied or neutralized by component (B).

It is generally a characteristic feature of the flame retardant composition of the invention that it contains that the salt of components (A) and (B) in an amount in the range from 1% to 100% by weight, preferably in the range from 30% to 100% by weight, based on the sum total of all components of the flame retardant composition. In a further embodiment, the flame retardant composition of the invention consists to an extent of 100% by weight of the salt of components (A) and (B).

The present invention further relates to a process for producing the above-described flame retardant composition. It is a feature of the process that components (A) and (B) are dispersed in a suitable liquid medium and converted at a temperature in the range from 60° C. to 120° C., preferably in the range from 90° C. to 100° C., over a period of 1 to 9 hours, preferably over a period of 5 to 7 hours. The reaction mixture obtained is then filtered, washed with a suitable solvent such as toluene, isopropanol, dioxane or water, and dried.

The suitable liquid medium is generally selected from the group of toluene, isopropanol, dioxane and water. In a preferred embodiment of the invention, the liquid medium is toluene.

Components (A) and (B) are known to those skilled in the art. Components (A) can be prepared by pyrolytic means, for example as described in U.S. Pat. No. 4,129,588 or EP 0 710 664 B1.

The present invention further relates to the use of the flame retardant composition for rendering polymers or polymer blends flame-retardant. The invention further relates to polymer molding compounds that have been rendered flame-retardant and comprise this flame retardant composition. The polymer or polymer substrate may be any of a large number of polymer types including polyolefins, polyesters, polyamides and ABS polymers. The polymers are more preferably selected from polyolefins such as polyethylenes and polypropylenes, polyesters, polyamides, polystyrenes, ABS polymers, polyvinyl chlorides, polyvinyl acetates, polyureas, polyacrylonitriles, phenol resins, melamine-formaldehyde resins and epoxy resins or mixtures of two or more thereof.

The effective flame-retardant amount of the flame retardant composition of the invention is that which is required to exhibit flame-retardant efficacy. This is measured by one of the standard methods which are used for assessment of flame retardancy. These include the NFPA 701 Standard Methods of Fire Tests for Flame Propagation of Textiles and Films 1989 and 1996 editions; the electro-UL 94 Test for Flammability of Plastic Materials for Parts in Devices and Appliances, 5th edition, 29 Oct. 1996, limiting oxygen index (LOI), ASTM-D 2863 and Cohen calorimetry ASTM E-1354. In addition, it is possible to employ the standards for buildings (DIN 4102 B1) and for motor vehicles (MVSS 302) to examine the flame-retardant properties of the flame retardant composition of the invention.

The present invention thus also relates to a composition comprising:

(a) one or more polymer(s) and

(b) an effective flame-retardant amount of the flame retardant composition of the invention.

The effective flame-retardant amount of the flame retardant composition of the invention is generally 0.1% to 30% by weight, more preferably 5% to 25% by weight, based on the polymer.

The flame-retardant polymers comprising the flame retardant compositions of the invention can be produced by known methods, for example by mixing the additives cited and optionally further additives with the polymer using equipment such as calenders, mixers, kneaders, extruders and the like. The additives can be added individually or in blends with one another. It is also possible to use what are called masterbatches. In such operations, it is possible to use the polymer, for example, in the form of powders, granules, solutions or suspensions or in the form of latices. The polymer compositions rendered flame-retardant that are thus obtained can be converted to the desired form by known methods. Such methods are, for example, calendering, extrusion, injection molding, spray coating, knife coating, spinning, compression melting, rotary casting, thermoforming or extrusion blowing. The finished polymer that has been rendered flame-retardant can also be processed to give foamed articles. The polymer articles produced in this way are, for example, fibers, films or foils, molded articles and foamed shaped bodies.

The present invention is elucidated in detail hereinafter with reference to the nonlimiting examples and comparative examples which follow.

### 1. PREPARATION EXAMPLE

### Preparation of dimelaminium pyromethanephosphonate

40.8 kg of pyromethanephosphonic acid and 59.2 kg of melamine are stirred in a nitrogen-inertized 800 1 reactor in 300 kg of toluene at 110° C. over a period of 3 hours. Thereafter, the solids are removed by means of a filter/filter press and then washed with 50 kg of toluene. After drying, 100 kg of dimelaminium pyromethanephosphonate are obtained (yield>99%). Phosphorus: 14.6%; nitrogen: 36.5%; theory: phosphorus: 14.6%; nitrogen: 39.6%.

### 2. EXAMPLES AND COMPARATIVE EXAMPLES

To illustrate the invention, LD polyethylene films (made from Lupolen® 1800) to which different flame-retardant compositions have been added are produced:

### Production of the Powder Mixtures:

An exact amount of Lupolen® 1800 polyethylene powder and powder mixtures of the flame retardants are blended and homogenized with the aid of a laboratory mixer.

### Production of the Films:

The films are produced on a Brabender 19/25D single-screw extruder at 30 rpm and various temperatures using a film mold to give films having a width of 100 mm and a thickness of 500 μm. The powder mixtures are supplied manually via a funnel. The mold used is a 100×0.5 mm wide strip die head; the draw-off belt is adjusted so as to achieve a film width of 90 mm and a thickness of 0.50 mm. The films are cooled down under air.

### Assessment of Efficacy:

The efficacy of the flame retardants was examined visually and in accordance with DIN 4102-B2. For this purpose, the films were clamped vertically in a combustion chamber. The flames were applied to the surface with a flame height of 20 mm and a flame angle of 45° . These DIN satisfy DIN 4102-B2 if the upper measurement mark on the flame test specimen is not reached by the tip of the flame or the flame is extinguished of its own accord.

The flame retardant of the invention used was the dimelaminium pyromethanephosphonate prepared in example 1 with the commercial available flame retardants melamine methanephosphonate (AFLAMMIT® PCO 800), melamine pyrophosphate, melamine polyphosphate (Melapur® 200) and diethylaluminum phosphinate “Depal” (Exolit® OP 930):

| | | % by wt. of | Films | Films | B2 flame |
|---|---|---|---|---|---|
| | % by wt. | Lupolen ® | (extruded | (extruded | test |
| | of FSM | 1800 | at 230° C.) | at 280° C.) | result |
| Example 1 | 5 | 95 | ok | ok | pass |
| Melamine | 5 | 95 | ok | holes | pass |
| methanephosphonate | | | | | |
| “AFLAMMIT ® PCO | | | | | |
| 800” | | | | | |
| Melamine | 5 | 95 | ok | ok | burns |
| pyrophosphate | | | | | |
| Melamine | 5 | 95 | ok | ok | burns |
| polyphosphate | | | | | |
| “Melapur ® 200” | | | | | |
| Diethylaluminum | 5 | 95 | ok | ok | burns |
| phosphinate | | | | | |
| “Exolit ® OP 930” | | | | | |

As can be inferred from the results shown in the table, the dimelaminium pyromethanephosphonate of the invention is superior to the flame retardants known from the prior art. While melamine methanephosphonate (AFLAMMIT® PCO 800) is comparable in terms of its flame retardancy properties to those of dimelaminium pyromethanephosphonate, all the other flame retardant compositions show a worse flame test result according to DIN 4102-B2. In direct comparison with melamine methanephosphonate (AFLAMMIT® PCO 800), it is noticeable that the flame retardant compositions of the invention have distinct advantages particularly at processing temperatures in the region of 280° C. While the film modified with the flame retardant of the invention does not have any holes, the film modified with melamine methanephosphonate (AFLAMMIT® PCO 800) has visual defects in the form of holes.

1. Flame retardant composition comprising a salt of components (A) and (B), wherein
(A) is at least one pyrophosphonic acid derivative of the general formula (I); 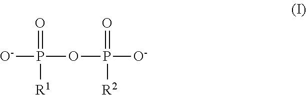
where R¹ and R² are each independently a substituted or unsubstituted, straight-chain or branched alkyl group having 1 to 4 carbon atoms, substituted or unsubstituted benzyl or substituted or unsubstituted phenyl, and
(B) is selected from the group consisting of melamine, melam, melem, melon, ammeline, ammelide, 2-ureido-melamine, substituted or unsubstituted heptazine, guanylurea, acetoguanamine, benzoguanamine and diaminophenyltriazine or two or more of these compounds.

2. Flame retardant composition according to claim 1, characterized in that the pyrophosphonic acid derivative is an alkylpyrophosphonic acid derivative of the formula (II)

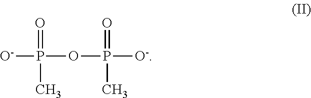

3. Flame retardant composition according to claim 1, characterized in that component (B) is melamine.

4. Flame retardant composition according to claim 1, characterized in that the salt of components (A) and (B) is present in an amount in the range from 1% to 100% by weight, based on the sum total of all the components of the flame retardant composition.

5. Process for producing the flame retardant composition according to any of claim 1, characterized in that components (A) and (B) are dispersed in a suitable liquid medium and converted at a temperature in the range from 60° C. to 120° C. over a period of 1 to 6 hours.

6. Use of a flame retardant composition according to claim 1 for rendering polymers flame-retardant.

7. Use according to claim 6, wherein the polymer is selected from polyolefins such as polyethylenes and polypropylenes, polyesters, polyamides, polystyrenes, ABS polymers, polyvinyl chlorides, polyvinyl acetates, polyureas, polyacrylonitriles, phenol resins, melamine-formaldehyde resins and epoxy resins or mixtures of two or more thereof.

8. Use according to claim 6, characterized in that the flame retardant composition is used in a concentration of 1% to 30% by weight, based on the polymer.

9. Polymer molding compound that has been rendered flame-retardant and comprises a flame retardant composition according to claim 1.

10. Method of imparting flame-retardant properties to a polymer, wherein the method comprises the addition of the flame retardant composition according to claim 1 to the polymer.

11. Polymer comprising a flame retardant composition according to claim 1.

12. Polymer according to claim 11, selected from polyolefins such as polyethylenes and polypropylenes, polyesters, polyamides, polystyrenes, ABS polymers, polyvinyl chlorides, polyvinyl acetates, polyureas, polyacrylonitriles, phenol resins, melamine-formaldehyde resins and epoxy resins or mixtures of two or more thereof.

13. Articles comprising the polymer according to claim 1 and selected from fibers, films, foils, molded articles and foamed shaped bodies.
